(19) United States
(12) Patent Application Publication
(10) Pub. No.: US 2025/0143819 A1
(43) Pub. Date: May 8, 2025

(54) TECHNIQUES FOR REPOSITIONING A COMPUTER-ASSISTED SYSTEM WITH MOTION PARTITIONING

(71) Applicant: INTUITIVE SURGICAL OPERATIONS, INC., Sunnyvale, CA (US)

(72) Inventors: Dinesh RABINDRAN, San Jose, CA (US); Simon P. DIMAIO, San Carlos, CA (US); Omid MOHARERI, San Francisco, CA (US)

(21) Appl. No.: 18/837,277

(22) PCT Filed: Feb. 21, 2023
(86) PCT No.: PCT/US2023/013536

§ 371 Date: Aug. 9, 2024

Related U.S. Application Data
(60) Provisional application No. 63/312,765, filed on Feb. 22, 2022.

(51) Int. Cl. A61B 34/37; A61B 34/20; A61B 34/35; G06T 7/70

(52) U.S. Cl. CPC A61B 34/37; A61B 34/20; A61B 34/35; G06T 7/70; A61B 2034/2055

(57) ABSTRACT

Techniques for repositioning a computer-assisted system include the following. The computer-assisted system comprises a repositionable structure system, the repositionable structure system comprising a plurality of links coupled by a plurality of joints, and a control unit communicably coupled to the repositionable structure system. The control unit is configured to: determine a target pose of a system portion of the computer-assisted system, determine a current pose of the system portion, determine a motion for the repositionable structure system based on a difference between the target pose and the current pose, the motion including a first component in a first direction, determine a partitioning of the first component into a plurality of partitions, and cause a first movement of a first joint set to achieve a first partition and a second movement of a second joint set to achieve a second partition.

### RELATED APPLICATIONS

This application claims the benefit to U.S. Provisional Application No. 63/312,765, filed Feb. 22, 2022, and entitled “TECHNIQUES FOR REPOSITIONING A COMPUTER-ASSISTED SYSTEM WITH MOTION PARTITIONING,” which is incorporated by reference herein.

### TECHNICAL FIELD

The present disclosure relates generally to electronic systems and more particularly relates to repositioning a computer-assisted system with motion partitioning.

### BACKGROUND

Computer-assisted electronic systems are being used more and more often. This is especially true in industrial, entertainment, educational, and other settings. As a medical example, the medical facilities of today have large arrays of electronic systems being found in operating rooms, interventional suites, intensive care wards, emergency rooms, and/or the like. Many of these electronic systems may be capable of autonomous or semi-autonomous motion. It is also known for personnel to control the motion and/or operation of electronic systems using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

Oftentimes, an electronic system needs to be repositioned within a physical environment in order to give the electronic system access to a worksite. Returning to the medical example, the electronic system may comprise a medical system that needs to be repositioned to provide access to an interior anatomy of a patient. The physical environment can include obstacles, such as the patient, an operating table, other equipment, fixtures such as lighting fixtures, personnel, and/or the like, that should be avoided when repositioning the medical system. Conventionally, repositioning an electronic system can require a team of two or more operators to communicate verbally and/or through gestures to move the electronic system while avoiding obstacles. However, the operators can be inexperienced or otherwise benefit from assistance to reposition the electronic system properly while avoiding obstacles. In the medical context, observing and reacting to obstacles also distracts from the attention operators may need to pay to other stimuli such as patient status and location, and tasks being performed by others.

Accordingly, improved techniques for repositioning a computer-assisted system are desirable.

### SUMMARY

Consistent with some embodiments, a computer-assisted system includes a repositionable structure system and a control unit. The repositionable structure system includes a plurality of links coupled by a plurality of joints. The control unit is communicably coupled to the repositionable structure system. The control unit is configured to: determine a target pose of a system portion of the computer-assisted system, the target pose of the system portion comprising at least one parameter selected from the group consisting of: a target position of the system portion and a target orientation of the system portion, determine a current pose of the system portion, the current pose of the system portion comprising at least one parameter selected from the group consisting of: a current position of the system portion and a current orientation of the system portion, determine a motion for the repositionable structure system based on a difference between the target pose and the current pose, the motion including a first component in a first direction, determine a partitioning of the first component into a plurality of partitions, wherein a first partition of the plurality of partitions is associated with a first joint set of the plurality of joints, and a second partition of the plurality of partitions is associated with a second joint set of the plurality of joints, the first joint set differing from the second joint set, and cause a first movement of the first joint set to achieve the first partition and a second movement of the second joint set to achieve the second partition.

Consistent with some embodiments, a method for controlling a repositionable structure system, which includes a plurality of links coupled by a plurality of joints, includes determining a target pose of a system portion of a computer-assisted system, the target pose of the system portion comprising at least one parameter selected from the group consisting of: a target position of the system portion and a target orientation of the system portion. The method also includes determining a current pose of the system portion, the current pose of the system portion comprising at least one parameter selected from the group consisting of: a current position of the system portion and a current orientation of the system portion. The method further includes determining a motion for the repositionable structure system based on a difference between the target pose and the current pose, the motion including a first component in a first direction. In addition, the method includes determining a partitioning of the first component into a plurality of partitions, wherein a first partition of the plurality of partitions is associated with a first joint set of the plurality of joints, and a second partition of the plurality of partitions is associated with a second joint set of the plurality of joints, the first joint set differing from the second joint set. The method further includes causing a first movement of the first joint set to achieve the first partition and a second movement of the second joint set to achieve the second partition.

Other embodiments include, without limitation, one or more non-transitory machine-readable media including a plurality of machine-readable instructions, which when executed by one or more processors, are adapted to cause the one or more processors to perform any of the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram including an example of a computer-assisted system, according to various embodiments.

FIG. 2 depicts an illustrative configuration of a sensor system, according to various embodiments.

FIG. 3 illustrates the control module of FIG. 1 in greater detail, according to various embodiments.

FIG. 4 illustrates a simplified diagram of a method for determining a motion of a repositionable structure in a linear direction when a computer-assisted system is being repositioned, according to various embodiments.

FIG. 5 illustrates a simplified diagram of a method for determining a motion of a repositionable structure system in an angular direction when a computer-assisted system is being repositioned, according to various embodiments.

FIG. 6 illustrates a simplified diagram of a method for partitioning motion along a direction of interest, according to various embodiments.

FIG. 7 illustrates an example of determining a motion of a repositionable structure system in a linear direction to avoid obstacles, according to various embodiments.

FIG. 8 illustrates an example partitioning of the motion of FIG. 7, according to various embodiments.

FIG. 9 illustrates an example feasible partitioning solution space for the example partitioning of FIG. 8, according to various embodiments.

FIG. 10 illustrates an example of determining a motion of a repositionable structure system in an angular direction to approach an object, according to various embodiments.

### DETAILED DESCRIPTION

This description and the accompanying drawings that illustrate inventive aspects, embodiments, embodiments, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “pose” refers to the position, the orientation, or the position and the orientation combined, of an element or a portion of an element. As used herein, the term “shape” refers to a set positions or orientations measured along an element. As used herein, and for an element or portion of an element. e.g. a device (e.g., a computer-assisted system or a repositionable arm), the term “proximal” refers to a direction toward the base of the system or device of the repositionable arm along its kinematic chain, and the term “distal” refers to a direction away from the base along the kinematic chain.

Aspects of this disclosure are described in reference to computer-assisted systems, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci R: Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci R: Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

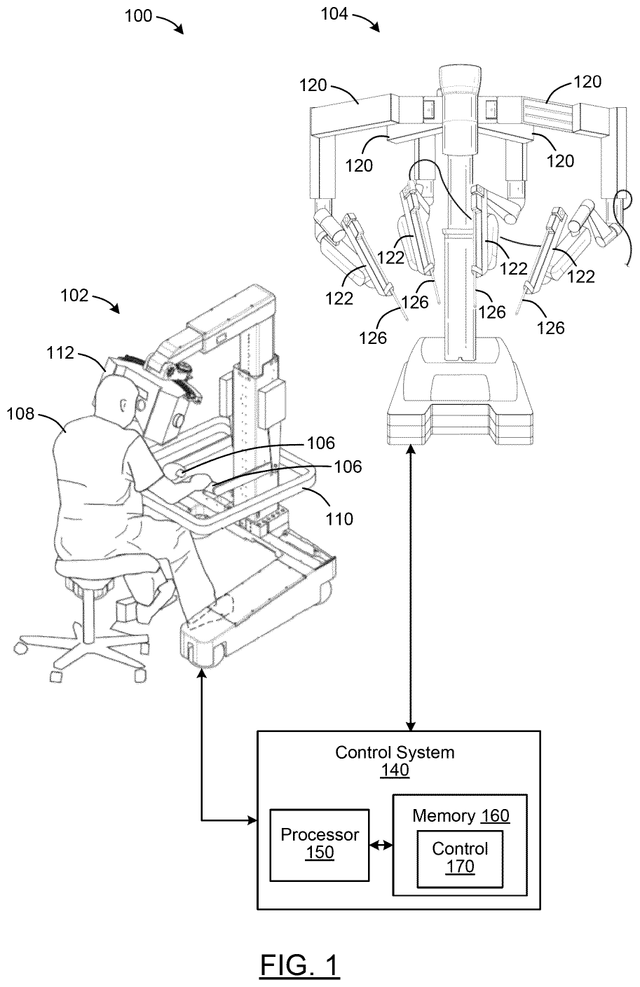

FIG. 1 is a simplified diagram of an example computer-assisted system, according to various embodiments. In some examples, the computer-assisted system is a teleoperated system 100. In medical examples, teleoperated system 100 can be a teleoperated medical system such as a surgical system. As shown, teleoperated system 100 includes a follower device 104 that may be teleoperated by being controlled by one or more leader devices (also called “leader input devices” when designed to accepted external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 which are designed to be contacted and manipulated by an operator 108. For example, workstation 102 can comprise one or more leader input devices 106 for use by the hands, the head, or some other body part of operator 108. Leader input devices 106 in this example are supported by workstation 102 and can be mechanically grounded. In some embodiments, an ergonomic support 110 (e.g., forearm rest) can be provided on which operator 108 can rest his or her forearms. In some examples, operator 108 can perform tasks at a worksite near follower device 104 during a procedure by commanding follower device 104 using leader input devices 106.

A display unit 112 is also included in workstation 102. Display unit 112 can display images for viewing by operator 108. Display unit 112 can be moved in various degrees of freedom to accommodate the viewing position of operator 108 and/or to optionally provide control functions as another leader input device. In the example of teleoperated system 100, displayed images can depict a worksite at which operator 108 is performing various tasks by manipulating leader input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

When using workstation 102, operator 108 can sit in a chair or other support in front of workstation 102, position his or her eyes in front of display unit 112, manipulate leader input devices 106, and rest his or her forearms on ergonomic support 110 as desired. In some embodiments, operator 108 can stand at the workstation or assume other poses, and display unit 112 and leader input devices 106 can be adjusted in position (height, depth, etc.) to accommodate operator 108.

In some embodiments, the one or more leader input devices 106 can be ungrounded (ungrounded leader input devices being not kinematically grounded, such as leader input devices held by the hands of operator 108 without additional physical support). Such ungrounded leader input devices can be used in conjunction with display unit 112. In some embodiments, operator 108 can use a display unit 112 positioned near the worksite, such that operator 108 manually operates instruments at the worksite, such as a laparoscopic instrument in a surgical example, while viewing images displayed by display unit 112.

Teleoperated system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126.

In various embodiments, one or more of instruments 126 can include an imaging device for capturing images (e.g., optical cameras, hyperspectral cameras, ultrasonic sensors, etc.). For example, one or more of instruments 126 could be an endoscope assembly that includes an imaging device, which can provide captured images of a portion of the worksite to be displayed via display unit 112.

In some embodiments, the manipulator arms 120 and/or instrument assemblies 122 can be controlled to move and articulate instruments 126 in response to manipulation of leader input devices 106 by operator 108, and in this way “follow” through teleoperation the leader input devices 106. This enables the operator 108 to perform tasks at the worksite using the manipulator arms 120 and/or instrument assemblies 122. Manipulator arms 120 and follower device 104 are examples of repositionable structures on which instruments such as manipulating instruments or and/or imaging instruments including imaging devices can be mounted. For a surgical example, the operator 108 could direct follower manipulator arms 120 to move instruments 126 to perform surgical procedures at internal surgical sites through minimally invasive apertures or natural orifices.

As shown, a control system 140 is provided external to workstation 102 and communicates with workstation 102. In other embodiments, control system 140 can be provided in workstation 102 or in follower device 104. As operator 108 moves leader input device(s) 106, sensed spatial information including sensed position and/or orientation information is provided to control system 140 based on the movement of leader input devices 106. Control system 140 can determine or provide control signals to follower device 104 to control the movement of manipulator arms 120, instrument assemblies 122, and/or instruments 126 based on the received information and operator input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1002.11, DECT, Wireless Telemetry, and/or the like).

Control system 140 can be implemented on one or more computing systems. One or more computing systems can be used to control follower device 104. In addition, one or more computing systems can be used to control components of workstation 102, such as movement of a display unit 112.

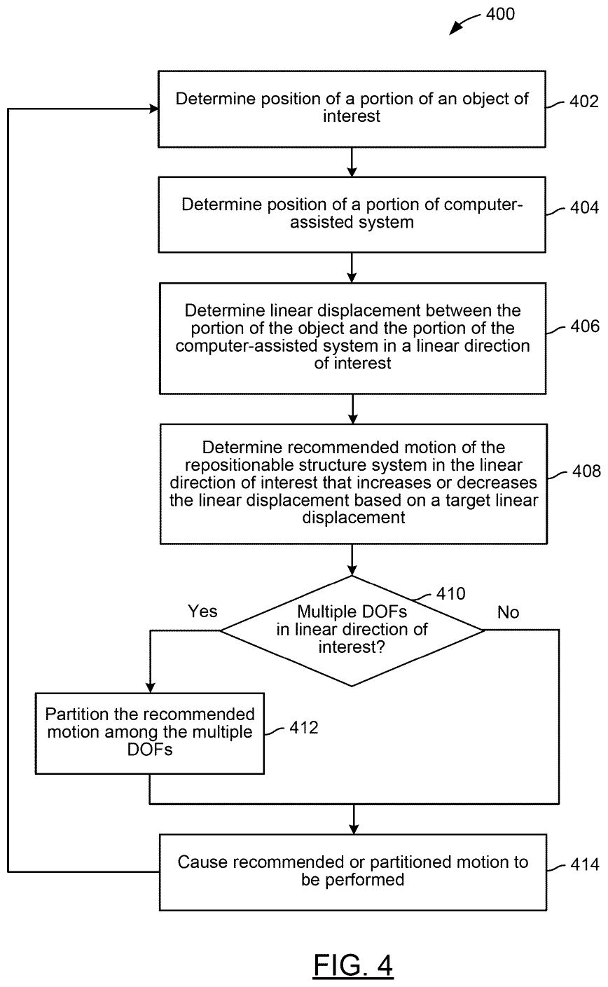

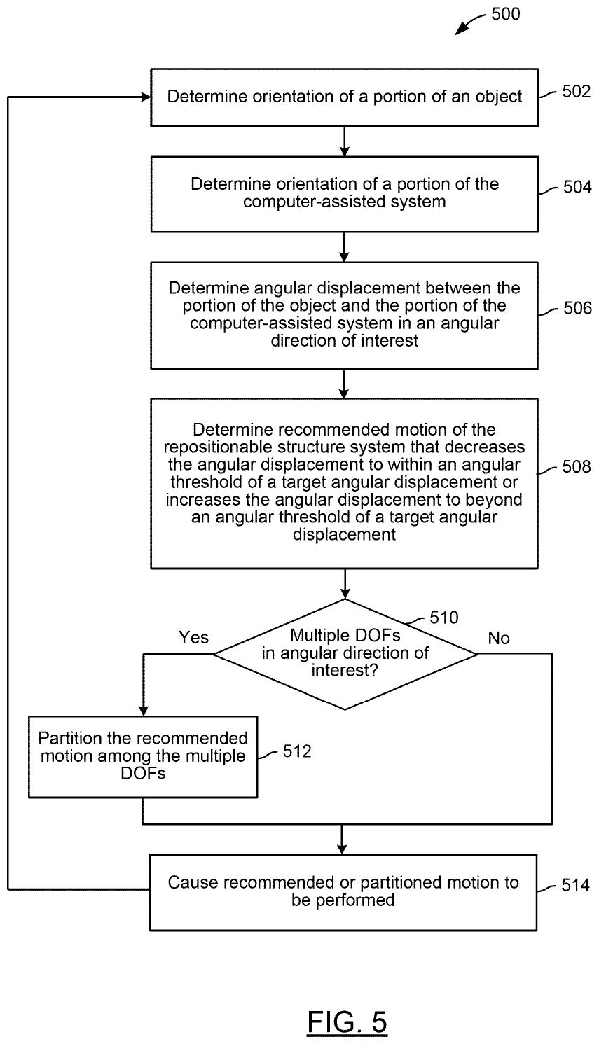

As shown, control system 140 includes a processor 150 and a memory 160 storing a control module 170). In some embodiments, control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, any other memory chip or cartridge, punch cards, paper tape, any other physical medium with patterns of holes, etc.), a communication interface (e.g., Bluetooth interface, infrared interface, network interface, optical interface, etc.), and numerous other elements and functionalities. The non-persistent storage and persistent storage are examples of non-transitory, tangible machine readable media that can include executable code that, when run by one or more processors (e.g., processor 150)), may cause the one or more processors to perform one or more of the techniques disclosed herein, including the processes of methods 400, 500, and/or 600 and/or the processes of FIGS. 4, 5, and/or 6, described below. In addition, functionality of control module 170 can be implemented in any technically feasible software and/or hardware in some embodiments.

Each of the one or more processors of control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, key board, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci R Surgical System, commercialized by Intuitive Surgical, Inc. of Sunnyvale, California, U.S.A. Embodiments on da Vinci R Surgical Systems are merely examples and are not to be considered as limiting the scope of the features disclosed herein. For example, different types of teleoperated systems having follower devices at worksites, as well as non-teleoperated systems, can make use of features described herein.

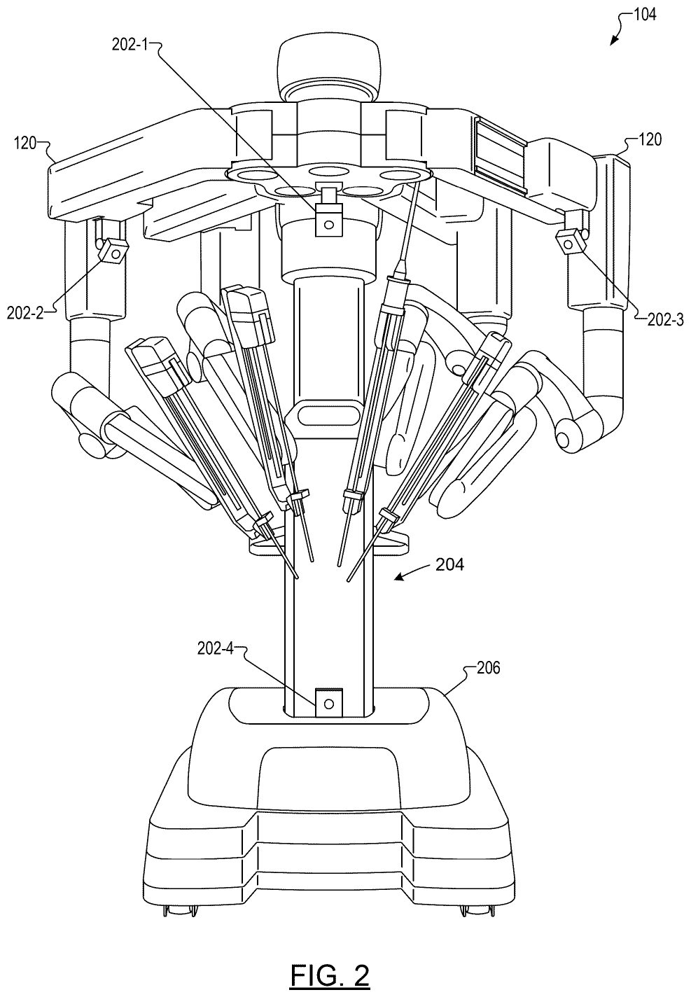

FIG. 2 depicts an illustrative configuration of a sensor system, according to various embodiments. As shown, imaging devices 202 (imaging devices 202-1 through 202-4) are attached to portions of follower device 104. Although described herein with respect to imaging devices as a reference example, in some embodiments, a sensor system can include any technically feasible sensors, such as monoscopic and stereoscopic optical systems, ultrasonic systems, depth cameras such as cameras using time-of-flight sensors, LIDAR sensors, etc. that are mounted on a computer-assisted system and/or elsewhere. For example, one or more sensors could be mounted on a base, on a set-up structure 204, and/or on one or more manipulator arms 120 of follower device 104. As another example, one or more sensors could be worn by an operator or mounted to a wall, a ceiling, the floor, or other equipment such as tables or carts.

Illustratively, imaging device 202-1 is attached to set-up structure 204 of follower device 104, imaging device 202-2 is attached to manipulating arm 120-1 of follower device 104, imaging device 202-3 is attached to manipulating arm 120-4 of follower device 104, and imaging device 202-4 is attached to a base 206 of follower device 104. In implementations in which follower device 104 is positioned proximate to a patient (e.g., as a patient side cart), placement of imaging devices 202 at strategic locations on follower device 104 provides advantageous imaging viewpoints proximate to a patient and areas around a worksite where a surgical procedure is to be performed on the patient.

In certain embodiments, components of follower device 104 (or other robotic systems in other examples) can have redundant degrees of freedom that allow multiple configurations of the components to arrive at the same output position and/or output orientation of an end effector attached to the components (e.g., an instrument connected to a manipulator arm 120). Accordingly, control system 140 can direct components of follower device 104 to move without affecting the position and/or orientation of an end effector attached to the components, thereby allowing for repositioning of components to be performed without changing the position and/or orientation of an end effector attached to the components.

The placements of imaging devices 202 on components of follower device 104 as shown in FIG. 2 are illustrative. Additional and/or alternative placements of any suitable number of imaging devices 202 and/or other sensors on follower device 104, other components of teleoperated system 100, and/or other components (not shown) located in proximity to the follower device 104 can be used in sensor systems in other embodiments. Imaging devices 202 and/or other sensors can be attached to components of follower device 104, other components of teleoperated system 100, and/or other components in proximity to follower device 104 in any suitable way. Additional computer-assisted systems including sensor systems that include sensors are described in International Patent Application No. PCT/US2021/059213, filed Nov. 12, 2021, and titled “Visibility Metrics in Multi-View Medical Activity Recognition Systems and Methods,” which is hereby incorporated by reference herein.

Repositioning a Computer-Assisted System with Motion Partitioning

A computer-assisted system can be repositioned within a physical environment while reducing the risk of collisions with obstacles, moving one or more joints closer to the center(s) of their respective ranges of motion, selectively operating joints to improve responsiveness, dexterity, power consumption, etc. In some embodiments, repositioning the computer-assisted system includes partitioning motion in linear and/or angular direction(s) of interest among one or multiple degrees of freedom (DOFs) provided by different joints of a repositionable structure system of the computer-assisted system.

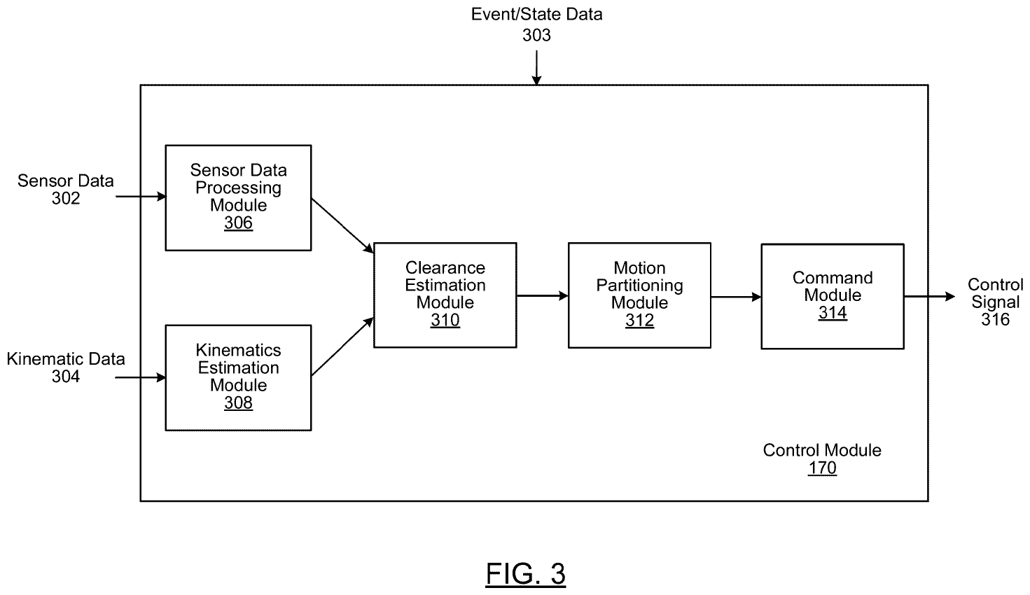

FIG. 3 illustrates control module 170 of FIG. 1 in greater detail, according to various embodiments. As shown, control module 170 includes a sensor data processing module 306, a kinematics estimation module 308, a clearance estimation module 310, a motion partitioning module 312, and a command module 314. Sensor data processing module 306 receives sensor data 302 and determines the positions and/or orientations of objects, and/or portions thereof, based on sensor data 302. Examples of sensor data 302 and sensors for collecting sensor data 302 are described above in conjunction with FIG. 2. Examples of objects and/or portions of objects in the medical context include a patient, a top of a patient, an operator, other personnel, a cannula, a fixture, an operating table, equipment (e.g., stands, patient monitoring equipment, drug delivery systems, imaging systems, etc.), other obstacles, etc. and/or portions thereof that are in a direction of motion of follower device 104. In some embodiments, sensor data processing module 306 can employ point cloud, object detection, object segmentation, and/or part segmentation techniques to determine the positions and/or orientations of objects and/or portions thereof. Additional and/or alternative techniques for detecting objects and/or portions thereof using registered sensors are described in International Patent Application No. International Application Publication No. WO 2021/097332, filed Nov. 13, 2020, and titled “Scene Perception Systems and Methods,” which is hereby incorporated by reference herein.

Kinematics estimation module 308 receives kinematics data 304 associated with the joints of a repositionable structure of follower device 104. Given kinematics data 304, kinematics estimation module 308 uses one or more kinematic models of the repositionable structure, and optionally a three-dimensional (3D) model of follower device 104, to determine positions and/or orientations of one or more portions of follower device 104. Returning to the medical example, the positions and/or orientations of portion(s) of follower device 104 can include the heights of cannula mounts or other portions of follower device 104, an overall height of follower device 104, horizontal positions of manipulator arms 120 or other portions of follower device 104, orientations of manipulator arms 120 or other portions of follower device 104, and/or the like. In some embodiments, kinematics data 304 is synchronized with sensor data 302 so that comparisons can be made between positions and/or orientations that are determined using both types of data corresponding to the same point in time.

Clearance estimation module 310 determines displacements, along one or more linear and/or angular directions of interest, between one or more portions of objects, and one or more portions of follower device 104 (or some other part of the computer-assisted system, such as some other part of the larger teleoperated system 100). Each displacement can be a directional vector that includes a magnitude and a direction. In the illustrated example, the positions and/or orientations of the portion(s) of object(s) needed for the displacement determination are output by sensor data processing module 306, and the positions and/or orientations needed of the follower device 104 are output by kinematics estimation module 308. In some embodiments, clearance estimation module 310 can determine linear and/or angular displacements between bounding regions around portion(s) of object(s) and bounding regions around portion(s) of a computer-assisted system. In such cases, each bounding region can be a convex hull, bounding box, mesh, one or more maxima points, one or more minima points, or other approximation. Subsequent to determining the linear and/or angular displacements, clearance estimation module 310 determines one or more recommended motions of a repositionable structure system that increases (repulsive cases) or decreases (attractive cases) each of the determined linear and/or angular displacements based on a target linear and/or angular displacement. A repositionable structure system can include a single repositionable structure, or multiple repositionable structures. For example, a repositionable structure system can include one or more repositionable structures of follower device 104, and/or of other devices. Examples of other devices include robotic operating tables, robotic devices with one or more manipulator arms (other than the follower device 104), etc.

In some embodiments, the recommended motion can be determined by the following technique. First, determine a current pose, which can include a current position and/or orientation of the repositionable structure system or a portion thereof. Then, determine the recommended motion based on a difference between the current pose and a target pose of the repositionable structure system. The target pose is associated with the target linear and/or angular displacement.

In repulsive cases, the linear and/or angular displacement can be increased beyond a threshold of a target linear and/or angular displacement. In such cases, the target linear and/or angular displacement can include a clearance linear and/or angular displacement required to avoid an object. In some embodiments, the target linear and/or angular displacement can also include a tolerance factor, such as a safety factor. For example, the target linear and/or angular displacement could be a clearance linear and/or angular displacement plus a tolerance factor. In attractive cases, the linear and/or angular displacement can be decreased to be within a threshold linear and/or angular displacement. In such cases, the target linear and/or angular displacement can include the threshold linear and/or angular displacement, as well as a tolerance factor. In some embodiments, the tolerance factor and/or the target linear and/or angular displacement can vary depending on environmental features, operating modes, operating conditions, an operator preference that is automatically determined by the system (such as based on information about the operator or history of use), or manually input, etc. For example, in some embodiments, the tolerance factor can be different under different circumstances (e.g., depending on a type of follower device 104, operating mode, a procedure being performed, operator preference, etc.). As another example, in some embodiments, the tolerance factor can be computed based on an uncertainty in the vision-based estimates by sensor data processing module 306 and/or the kinematics-based position estimates by kinematics estimation module 308. For example, higher uncertainties can be accounted for using higher tolerance factors, and vice versa.

In some embodiments, the repulsive or attractive case can be chosen globally, based on the environmental feature (e.g. obstacles are repulsive and empty spaces are attractive), or in any other technically feasible manner. In some examples, an object may have both repulsive and attractive cases. Returning to the medical example, a patient could have a first, smaller linear and/or angular displacement threshold within which repulsion is used as well as a second, larger linear and/or angular displacement threshold outside of which attraction is used.

In some embodiments, determination of the motion to increase or decrease the linear and/or angular displacement based on the target linear and/or angular displacement can be initiated based on any technically feasible conditions. In some embodiments, the initiation can be triggered by event and/or system state data 303 associated with the computer-assisted system that is received in addition to sensor data 302 and kinematics data 304. For example, in some embodiments, the initiation can be based on a system mode change, which can be triggered by entering a certain zone. In such cases, the zone can have suitable shape. For example, the zone can be a spherical zone (e.g., a zone that is a given radius around a worksite), a cylindrical zone, a rectangular zone, a zone of irregular shape, etc. As another example, in some embodiments, the initiation can be based on the visibility of an object of interest and/or the confidence of a computer vision technique, such as an object segmentation confidence. As another example, in some embodiments, the initiation can be based on a linear and/or angular displacement from a target object. As a further example, the initiation can be by an operator, such as via a switch or other user input.

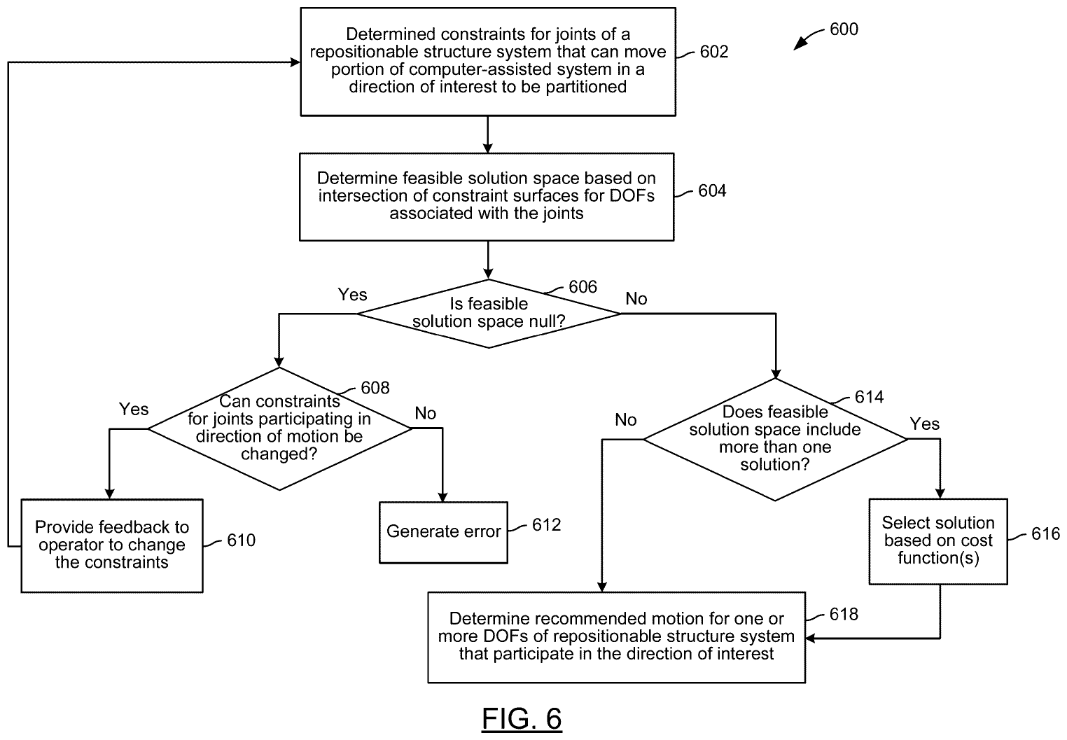

Motion partitioning module 312 performs motion partitioning to split the amount of linear and/or angular motion along each direction of interest between two joint sets, or among three or more joint sets, of a repositionable structure system. Each joint set can include one or more joints. As described, the repositionable structure system can include repositionable structure(s) of the follower device 104 and/or repositionable structure(s) of other device(s) (e.g., a patient side cart, additional repositionable device, a table, an imaging cart, etc.) in some embodiments. In such cases, the motion partitioning can split the amount of motion along each direction of interest between two or more joint sets in the repositionable structure(s) of the follower device 104 and/or the repositionable structur(es) of the other device(s). Then, the motion to be performed by the repositionable structure system can include motion of portion(s) of the repositionable structure(s) (e.g., a highest portion, a longest portion, a widest portion) of follower device 104 and/or motion of the repositionable structure(s) of other device(s) (e.g., an additional repositionable device, a patient-side cart, a table, an imaging cart, etc.), in at the least one of the directions of interest. The directions of interest can be in any spatial direction and defined using a coordinate system, such as the Cartesian or spherical coordinate system. When a Cartesian coordinate system is used, movement in a direction of interest can be defined with reference to one or a combination of Cartesian DOFs (e.g., translations along one or more linear degrees of freedom, with motion components along x, y, and/or z axes; and/or rotations in one or more rotational degrees of freedom, with motion about one or more axes defined by pairs of points located in the Cartesian coordinate system by x, y, and z values). Techniques for partitioning motion in a direction of interest are discussed in greater detail in conjunction with FIGS. 6, 8, and 9. In some embodiments, motion along any number of linear and/or angular directions of interest can be partitioned.

In some embodiments, motion partitioning module 312 can partition motion along a direction of interest into joint null-space motions that maintain an orientation and/or position of one or more components, points, or reference frames of interest. For example, the joint null-space motions can be used to help avoid obstacles while maintaining such orientation and/or position.

Command module 314 causes follower device 104 to move based on the motion partitioning output of motion partitioning module 312 or the recommended motion output of the clearance estimation module 310. In some embodiments, the repositionable structure of follower device 104 is moved automatically. In such cases, command module 314 can employ inverse kinematics to compute joint motions for subsystems of the repositionable structure system, or the entire repositionable structure system, that are needed to achieve the motion partitioning output or the recommended motion. Then, command module 314 can generate and transmit a control signal 316 that includes one or more commands to an actuator system of follower device 104 to cause joints of follower device 104 to move according to the determined joint motions.

In some cases, motion to be performed by a repositionable structure system along one or more directions of interest can include movement of portions of the repositionable structure system in a null-space of the portions of the repositionable structure system. In some embodiments, a speed of the motions being performed can vary according to any technically feasible criteria. The speed can be a target speed, maximum speed, or a minimum speed, in some embodiments. For example, in some embodiments, command module 314 can decrease the speed of motions as follower device 104 approaches a worksite or a target position/orientation. In such a case, the decrease can be according to a monotonic function, such as a piecewise linear function, a linear function, or a non-linear function. As another example, in some embodiments, the speed of a motion can be determined based on a type of obstacle being avoided. As a further example, in some embodiments, the speed of a motion can be determined based on multiple parameters, such as the linear and/or angular displacement of follower device 104 from a worksite or a target position/orientation in addition to a speed of follower device 104 towards the worksite or target position/orientation. As yet another example, in some embodiments, the speed of motions can be selectable by an operator who can also pause and resume the motions. In some embodiments, when partitioned motions are being executed, commands can be generated and transmitted to execute each motion concurrently in a coordinated fashion, serially in a pre-determined order, or any combination thereof.

In some other embodiments, command module 314 can generate prompts that are output for operator(s) to move one or more portions of follower device 104, either in conjunction with or in lieu of the automated commands described above. In some cases, the prompted motion of a repositionable structure system can include null-space motion as well.

In some embodiments, the above behaviors of sensor data processing module 306, kinematics estimation module 308, clearance estimation module 310, motion partitioning module 312, and/or command module 314 can be allowed, inhibited, stopped, and/or overridden in any technically feasible manner. For example, in some embodiments, when follower device 104 is being moved away from a worksite, previous motions that were performed can be reversed. As another example, in some embodiments, when follower device 104 is being moved away from a worksite, the repositionable structure of follower device 104 can be commanded to move into a pre-programmed storage configuration.

FIGS. 4 to 6 illustrate methods 400, 500, 600, and one or more of the processes of each of methods 400, 500, and 600 may be implemented, partially or entirely, in the form of executable code stored on one or more non-transitory, tangible, machine readable media that, when performed by one or more processors (e.g., the processor 150 in control system 140) may cause the one or more processors to perform one or more of the processes of the method. In some embodiments, any of the methods disclosed herein (e.g., methods 400, 500, 600) may be performed by one or more modules, such as control module 170. In various embodiments, each of methods 400, 500, and 600 may include fewer processes than shown, or additional processes not shown, in the respective figures. In some embodiments, one or more of the processes of any of the methods disclosed herein (e.g., methods 400, 500, 600) may be performed, at least in part, by one or more of the modules of control system 140.

FIG. 4 illustrates a simplified diagram of a method 400 for determining a recommended motion of a repositionable structure in a linear direction of interest when a computer-assisted system is being repositioned, according to various embodiments. As shown, method 400 begins at process 402, where the position of a portion of an object of interest is determined. In some embodiments, the position of the portion of the object can be determined based on sensor data and a machine learning or other computer vision technique, as described above in conjunction with FIG. 3. The position of the portion of the object can be determined in any technically feasible manner in some embodiments. For example, in some embodiments where the other object includes a repositionable structure, such that the portion is a part of the repositionable structure, the position of the portion of the object can be determined using kinematic data of the repositionable structure. As another example, in some embodiments, the position of the portion of the object can be determined using any suitable sensor data for locating and registering components relative to one another. As a specific example, the object and the computer-assisted system may in some instances not have a fixed, manually defined or input, or predictable geometric relationship, such as one based on a mechanical connection between them. In such instances, the object and the computer-assisted system (and/or portions thereof) can be registered to one another based on image data depicting the poses of the object and the computer-assisted system, laser ranging data, ultrasonic data. RFID or emitter-receiver data usable for locating or orienting components relative to each other, and/or based on any other suitable data. The registering establishes a relationship between the object and the computer-assisted system (and/or the portions thereof) so that the position of the portion of the object can be determined relative to the computer-assisted system. Although process 402 is described with respect to the position of a portion of an object for simplicity, in some embodiments, the positions of any number of portions of any number of objects can be determined.

At process 404, the position of a portion of the computer-assisted system is determined. In some embodiments, the position of the portion of the computer-assisted system of the object can be determined based on kinematic data and forward kinematics, one or more kinematic models of the follower device 104, and/or a 3D model of follower device 104, as described above in conjunction with FIG. 3. Alternatively or in addition, the portion of the object and the computer-assisted system (and/or portions thereof) can also be registered to each other so that the position of the portion of the computer-assisted system can be determined relative to the portion of the object. In various embodiments, the position of the portion of the computer-assisted system can be determined in any technically feasible manner. For example, in some embodiments, the position of portion of the computer-assisted system can be determined using a machine learning used in conjunction with computer vision techniques. As another example, in some embodiments, the position of the portion of the computer-assisted system can be determined using any suitable sensor data, including sensor data for locating and registering components relative to one another. Although process 404 is described with respect to the position of a portion of a computer-assisted system for simplicity, in some embodiments, the positions of any number of portions of a computer-assisted system can be determined and compared with the positions of any number of portions of objects.

At process 406, a linear displacement in a linear direction of interest between the portion of the object and the portion of the computer-assisted system is determined. Returning to the medical example, the linear direction could be the vertical direction, and the linear displacement could be a displacement between the height of a patient and the height of a cannula mount of follower device 104. As another example, the linear direction could be the horizontal direction, and the linear displacement could be a displacement between a base or other portion of follower device 104 and a patient.

At process 408, a recommended motion of a repositionable structure system is determined that increases (repulsive cases) or decreases (attractive cases) the linear displacement based on a target linear displacement. In some embodiments, the recommended motion can be determined by first determining a current pose, which can include a current position and/or orientation, of the repositionable structure system, or a portion thereof, and then determining the recommended motion based on a difference between the current pose and a target pose associated with the target linear displacement and the current pose, as described above in conjunction with FIG. 3. In some embodiments, the target linear displacement can include a tolerance factor. In some embodiments, the target linear displacement can vary depending on environmental features, a procedure being performed, an operating mode, operating conditions, an operator preference that is automatically determined or manually input, a type of follower device 104, an uncertainty in vision-based and/or kinematics-based position estimates, etc. and/or a combination thereof.

At process 410, when multiple DOFs/joints can move the portion of the computer-assisted system in the linear direction of interest, then at process 412, the recommended motion is partitioned among the multiple DOFs/joints. Method steps for partitioning motion along a direction of interest are described in greater detail below in conjunction with FIG. 6.

At process 414, the recommended or partitioned motion is caused to be performed. In some embodiments, causing the recommended or partitioned motion to be performed includes determining joint motions based on the recommended or partitioned motion and kinematics, and transmitting commands to an actuator system to cause joints of the repositionable system to move according to the joint motions. In some other embodiments, causing the recommended or partitioned motion to be performed includes generating prompts that are output to one or more operator(s), instructing the operator(s) to move one or more portions of the repositionable structure system, either in conjunction with or in lieu of automated commands to the actuator system, described above.

After the recommended or partitioned motion is caused to be performed at process 414, and assuming the computer-assisted system continues to need to be repositioned, method 400 returns to process 402.

FIG. 5 illustrates a simplified diagram of a method 500 for determining a motion of repositionable structure system in an angular direction of interest when a computer-assisted system is being repositioned, according to various embodiments. One or more of the processes 502-514 of method 500 may be implemented, at least in part, in the form of executable code stored on non-transitory, tangible, machine readable media that when run by one or more processors (e.g., the processor 150 in control system 140) may cause the one or more processors to perform one or more of the processes 502-514. In some embodiments, method 500 may be performed by one or more modules, such as control module 170. In some embodiments, method 500 may include additional processes, which are not shown. In some embodiments, one or more of the processes 502-514 may be performed, at least in part, by one or more of the modules of control system 140. In some embodiments, method 500 may be performed in addition to method 400, described above in conjunction with FIG. 4.

As shown, method 500 begins at process 502, where the orientation of a portion of an object of interest is determined. In some embodiments, the orientation of the portion of the object can be determined based on sensor data and a machine learning or other computer vision technique, as described above in conjunction with FIG. 3. The orientation of the portion of the object can be determined in any technically feasible manner in some other embodiments. For example, when the other object includes a repositionable structure, the orientation of the portion of the object can be determined using kinematic data in some embodiments. As another example, in some embodiments, the orientation of the portion of the object can be determined using any suitable sensor data for locating and registering components relative to one another. Although process 502 is described with respect to the orientation of a portion of an object for simplicity, in some embodiments, the orientations of any number of portions of any number of objects can be determined.

At process 504, the orientation of a portion of the computer-assisted system is determined. In some embodiments, the orientation of the portion of the computer-assisted system of the object can be determined based on kinematic data and forward kinematics, and optionally a 3D model of follower device 104, as described above in conjunction with FIG. 3. In such cases, the object and the computer-assisted system (and/or portions thereof) can also be registered to each other so that the orientation of the portion of the computer-assisted system can be determined relative to the object. The orientation of the portion of the computer-assisted system can be determined in any technically feasible manner in some other embodiments. For example, in some embodiments, the orientation of portion of the computer-assisted system can be determined using a machine learning or other computer vision technique. As another example, in some embodiments, the orientation of the portion of the computer-assisted system can be determined using any suitable sensor data, including sensor data for locating and registering components relative to one another. Although process 504 is described with respect to the orientation of a portion of a computer-assisted system for simplicity, in some embodiments, the orientations of any number of portions of a computer-assisted system can be determined and compared with the orientations of any number of portions of objects.

At process 506, an angular displacement is determined between the portion of the object and the portion of the computer-assisted system in an angular direction of interest. For example, the angular displacement could be the angle between a bearing angle of a midline of a table that is identified via a computer vision technique and a center or other aggregate orientation angle of a cluster of manipulator arms 120 about a support structure axis, measured in a base frame of reference of the follower device 104.

At process 508, a recommended motion of the repositionable structure system is determined that decreases (attractive cases) the angular displacement based on a target angular displacement or increases (repulsive cases) the angular displacement based on a target angular displacement. As a medical example, the target angular displacement could be a threshold angular displacement between the portion of the object and the portion of the computer-assisted system that is required to perform an operation. In some embodiments, the target angular displacement can vary depending on environmental features, a procedure being performed, an operating mode, operating conditions, an operator preference that is automatically determined or manually input, a type of follower device 104, an uncertainty in the vision-based and/or kinematics-based position estimates, etc. and/or a combination thereof. In some embodiments, the target angular displacement can include a tolerance factor.

At process 510, when multiple DOFs/joints can move the portion of the computer-assisted system in the angular direction of interest, then at process 512, the recommended motion is partitioned among the multiple DOFs/joints. Method steps for partitioning motion along a direction of interest are described in greater detail below in conjunction with FIG. 6.

At process 514, the recommended or partitioned motion is caused to be performed. In some embodiments, causing the recommended or partitioned motion to be performed includes determining joint motions based on the recommended or partitioned motion and kinematics, and transmitting commands to an actuator system to cause joints of the repositionable system to move according to the joint motions. In some other embodiments, causing the recommended or partitioned motion to be performed includes generating prompts that are output to one or more operator(s), instructing the operator(s) to move one or more portions of the repositionable structure system, either in conjunction with or in lieu of automated commands to the actuator system, described above.

After the recommended or partitioned motion is caused to be performed, and assuming the computer-assisted system continues to need to be repositioned, method 500 returns to process 502.

FIG. 6 illustrates a simplified diagram of a method 600 for partitioning motion along a direction of interest, according to various embodiments. In some embodiments, method 600 can be performed to partition motion at process 412 of method 400 and/or process 512 of method 500.

As shown, method 600 begins at process 602, where one or more constraints are determined for joints of a repositionable structure system that can move a portion of a computer-assisted system in a direction of interest to be partitioned. Constraints can be determined for any number of joints belonging to any number of joint sets. A joint set includes one or more joints. For example, constraints could be determined for a joint set associated with a manipulator arm 120 of follower device 104, a joint set associated with a support linkage of follower device 104, a joint set associated with an operating table, or a combination thereof, etc. In some embodiments, the constraints can include hardware-based constraints, environment-based constraints, kinematics-based constraints, and/or dynamics-based constraints. The hardware-based constraints can relate to physical limits of a repositionable structure system, such as range of motion (ROM) limits of joints of the repositionable structure system. The environment-based constraints can relate to obstacles in a direction of motion (e.g., operators, other personnel, fixtures, equipment, etc.), positioning and/or orienting of a worksite (e.g., the positioning of a patient or other target object for a given procedure, etc.), visibility/detectability of objects of interest, and/or characteristics of the environment. For example, an environment-based constraint could require that follower device 104 be kept at least a minimum distance away from a sterile zone. The kinematics-based constraints can relate to minimum linear and/or angular displacements between different portions of the repositionable structure (e.g., minimum displacements required for instrument removal/exchange clearance), the manipulability of manipulators of the repositionable structure system, etc. For example, manipulability constraints can be used to avoid ill-conditioned kinematics or manipulator configurations that overly limit the ability to manipulate a mounted instrument. The dynamics-based constraints can include constraints related to the inertia of a configuration of the repositionable structure system, closed and open loop bandwidths in a given configuration of the repositionable structure, etc.

In some embodiments, the constraints that are used to partition motion in a direction of interest can be updated in real time. In some embodiments, if different constraints exist for different portions of a repositionable structure system, an overall constraint is determined based on the constraints among all of the portions in the repositionable structure system. In some embodiments, the overall constraint can be determined as a worst-case (most restrictive) constraint among the portions of the repositionable structure system in the repulsive case or a best-case (least restrictive) constraint in the attractive case. In some other embodiments, the overall constraint can be determined as an average of the constraints for the portions of the repositionable structure system.

At process 604, a feasible solution space is determined based on an intersection of the constraint surfaces for DOFs associated with the joints. In some embodiments, the feasible solution space includes all of the DOFs/joints participating in the direction of interest.

At process 606, if the feasible solution space is null, then method 600 continues to process 608. The feasible solution space being null means that, in the current configuration of the repositionable structure system, the recommended motion in the direction of interest cannot be partitioned while satisfying the constraints.

At process 608, when the constraints for the joints of the repositionable structure system that participate in the direction of interest can be changed, then method 600 continues to process 610, where feedback is provided to an operator to change the constraints. In some embodiments, the feedback can include, for example, instructions and/or directions on moving an obstacle, manually reconfiguring the repositionable structure system, repositioning follower device 104 within the physical environment, etc., or a combination thereof to change the constraints.

Alternatively, when the constraints for the joints of the repositionable structure system that participate in the direction of interest cannot be changed, then method 600 continues to process 612, where an error is generated. In some embodiments, a computer-assisted system can be allowed to inadvertently collide with an object when the constraints for the joints that participate in a direction of interest cannot be changed. In such cases, an operator can also be warned of the collision.

Alternatively, when the feasible solution space is not null, then method 600 continues to process 614. At process 614, when the feasible solution space includes more than one solution, then method 600 continues to process 616, where a solution is selected based on one or more cost functions. The one or more cost functions are used to compare different solutions in the feasible solution space. Each of the solutions is associated with a partitioning candidate. In some embodiments, the one or more cost functions can be based on a displacement of joints to centers of ROMs, a measure of manipulability of links, a bandwidth of the DOFs, etc. or a combination thereof. For example, a cost function could be employed to favor solutions that minimize the displacement of joints to centers of ROMs of those joints. As another example, a cost function could be employed to favor solutions that use joints with high bandwidths when motions need to be performed more quickly. As another example, a cost function could be employed to partition motion along a direction of motion into joint null-space motions that maintain an orientation and/or position of on one or more components, points, or reference frames of interest.

At process 618, a recommended motion is determined for one or more DOFs of the repositionable structure system that participate in the direction of interest. In some embodiments, the recommended motion is determined based on the solution that is selected at process 616 and kinematics. For example, in some embodiments, inverse kinematics can be computed for subsystems of the repositionable structure system, or the entire repositionable structure system, in order to determine the recommended motion.

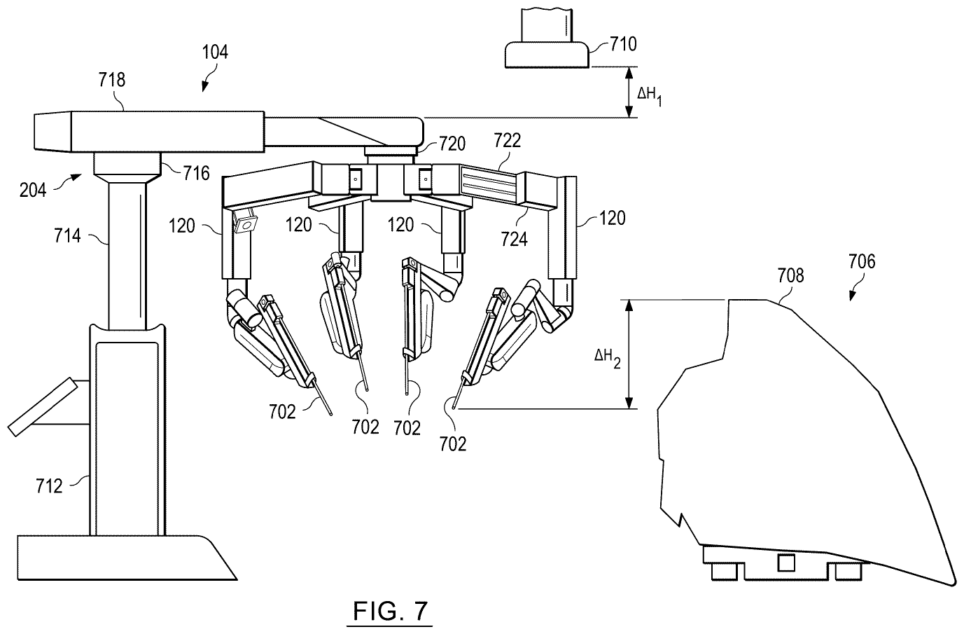

FIG. 7 illustrates an example of determining motion of a repositionable structure system in a linear direction to avoid obstacles, according to various embodiments. As shown, a repositionable structure of follower device 104 includes set-up structure 204 that supports a plurality of manipulator arms 120 that each include a cannula mount 702. Illustratively, the joints of set-up structure 204 that can move include a vertical shaft 714 out of a base 712 of support linkage 205, a rotational joint 716 with a vertical axis at the top of vertical shaft 714, a distal link 718, a rotational joint 720 that couples link 718 to a support structure 722 that in some implementations is referred to as an orienting platform, rotational joints 724 coupling corresponding manipulator arms 120 to support structure 722, and joints within each manipulator arm 120. In some embodiments, control module 170) can determine movements of a repositionable structure system that includes the repositionable structure of follower device 104 and/or repositionable structure(s) of other device(s) and provide 314s to an actuator system to achieve the determined movements, as described above in conjunction with FIGS. 3-5.

In the example of FIG. 7, control module 170 can determine movements of the repositionable structure of follower device 104 so as to avoid objects such as a patient 706 and an obstacle 710, which is shown as an overhead fixture, when follower device 104 is moved in a direction towards the patient 706. In the example shown, the obstacle 710 comprises an overhead lighting fixture: in other examples, the obstacle 710 can comprise other fixtures or instead be parts of other equipment or personnel, and be disposed overhead, underfoot, mid-height, floor, etc. Follower device 104 can be moved towards the patient 706 in any technically feasible manner, such as automatically, manually, or a combination thereof.

Illustratively, sensor data processing module 306 uses sensor data to determine a height of the patient 706. For example, in some embodiments, sensor data processing module 306 can employ a machine learning or other computer vision technique to segment and classify a point cloud generated from image data. The sensor data processing module 306 can also determine the height of the patient 706 from a highest point 708 that is classified as belonging to the patient 706.

In addition, kinematics estimation module 308 uses kinematic data and kinematics (and optionally a model of follower device 104) to determine the heights of cannula mounts 702 and/or the height of a lowest cannula mount 702 on manipulator arms 120 of follower device 104. As described, the kinematic data can correspond to the sensor data obtained at a same point in time so that positions determined based on such data can be compared with each other.

Similarly, sensor data processing module 306 can use sensor data to determine a height of the obstacle 710. In addition, kinematics estimation module 308 can use kinematic data to determine a height of set-up structure 204.

After the heights of patient 706, cannula mounts 702, set-up structure 204, and obstacle 710 are determined, clearance estimation module 310 determines (1) a displacement between the height of set-up structure 204 and the height of obstacle 710, shown as ΔH₁; and (2) a displacement between the height of cannula mounts 702 and the height of patient 706, shown as ΔH₂. It should be noted that the displacements can be negative if, for example, the height of patient 706 is above the height of cannula mounts 702.

Then, clearance estimation module 310 determines a recommended motion of the repositionable structure of follower device 104 that increases the displacement between the height of set-up structure 204 and the height of obstacle 710 based on a target displacement. As described, the target displacement can be a clearance displacement plus a tolerance factor in some embodiments. The target displacement can also be different for different circumstances, such as different environmental features, operating modes, operating conditions, an operator preference that is automatically determined or manually input, uncertainty in the vision-based and/or kinematics-based position estimates, etc. Increasing the displacement between the height of set-up structure 204 and the height of obstacle 710 based on the target displacement can help the follower device 104 to avoid collisions with obstacle 710 when the follower device 104 is being moved. It should be noted that no increase may be needed if the displacement between the height of set-up structure 204 and the height of obstacle 710 is greater than or equal to the target displacement.

Similarly, clearance estimation module 310 determines a recommended motion of the repositionable structure of follower device 104 that increases the displacement between the height of cannula mounts 702 and the height of patient 706 based on a target displacement. The same or a different target displacement can be used as is used for the displacement between the height of set-up structure 204 and the height of obstacle 710.

After the recommended motion is determined, motion partitioning module 312 can partition the recommended motion between multiple DOFs/joints that can move corresponding portion(s) of the repositionable structure of the follower device 104 in the vertical direction, such as vertical shaft 714 and joints in manipulator arms 120.

Although a repulsive case is shown for illustrative purposes in FIG. 6, displacements between portions of object(s) and portion(s) of follower device 104 can be decreased based on target displacements in attractive cases. Although an example with respect to the vertical direction is shown for illustrative purposes in FIG. 7, recommended motions of a repositionable structure system can also be determined for other linear direction(s), such as horizontally, to avoid and/or approach objects in a physical environment.

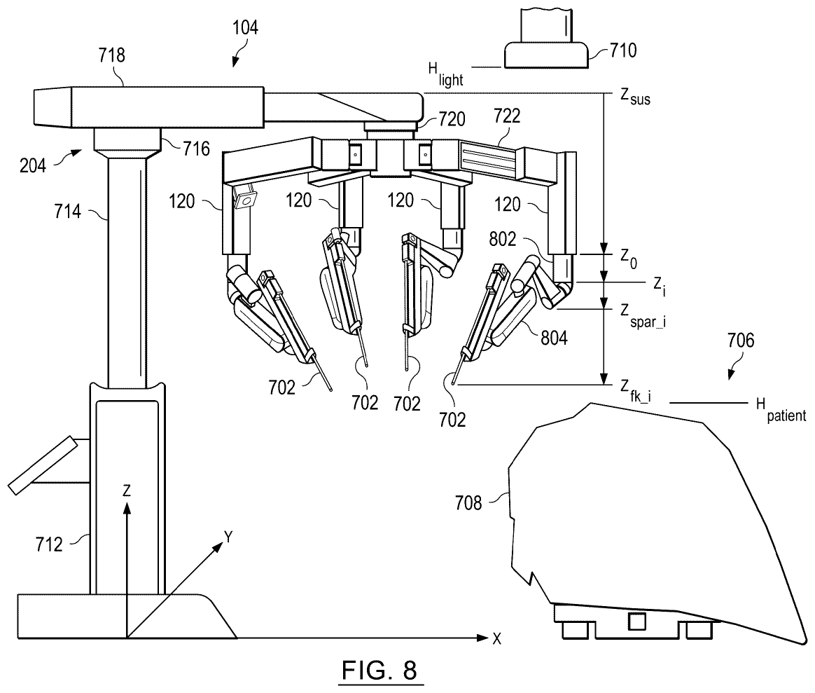

FIG. 8 illustrates an example partitioning of the recommended motion of FIG. 7, according to various embodiments. As shown, the recommended motion can be partitioned between DOFs provided by joints in set-up structure 204 and manipulator arms 120. Set-up structure 204 includes vertical shaft 714 that can be used to control a vertical height of set-up structure 204, which also affects the vertical heights of support structure 722 that is coupled to set-up structure 204, manipulator arms 120 that are coupled to support structure 722, cannula mounts 702 on manipulator arms 120, etc. In addition, manipulator arms 120 include joints that can be used to control the vertical heights of corresponding cannula mounts 702. In particular, each manipulator arm 120 includes a drop-link joint 802 that can be used to control a height of a cannula mount 702 with respect to support structure 722 and set-up structure 204. Motion in the vertical direction to avoid patient 706 and obstacle 710, as described above in conjunction with FIG. 7, can be partitioned among the DOFs provided by vertical shaft 714 and drop-link joints 802 of manipulator arms 120. For example, in some embodiments, the feasible partitioning solution space can be determined as the intersections of the following constraints:
1. Joint range of motion (ROM) constraints on set-up structure 204: 0<Zₛᵤₛ<Z_{sus_max}
2. Joint ROM on drop-link joints 802 of manipulator arms 120 that can change z_{spar_i}:−z_{i_max}<zᵢ<z_{i_max}
3. Patient 706 clearance constraint:
   a. Zₛᵤₛ−Z₀−zᵢ−z_{fk_i}−Hₚₐₜᵢₑₙₜ>c′₂, where c′₂ is a tolerance factor
   b. Zₛᵤₛ−zᵢ>c₂
4. Overhead constraint: Zₛᵤₛ<H_{light}−c₁
5. Instrument exchange constraint:
   a. Zₛᵤₛ−(Zₛᵤₛ−Z₀−zᵢ−z_{spar_i})>longest instrument length+c′₃, where c′₃ is a tolerance factor
   b. zᵢ>c₃

In the above constraints, Zₛᵤₛ is a height of set-up structure 204, which can vary between 0 at the floor and a maximum height of Z_{sus_max}: zᵢ is a displacement of a drop-link joint 802 on the i^{th} manipulator arm 120, which can vary between −z_{i_max} and z_{i_max}: Z₀ is a vertical displacement between the height of set-up structure 204 and a drop-down link: z_{fk_i} is a vertical displacement of a cannula mount 702 with respect to an end point of the i^{th} manipulator arm 120; z_{spar_i} is a vertical displacement of the top of a spar 804 of the i^{th} manipulator arm 120 with respect to the end point of a drop-link joint 802 for the i^{th} manipulator arm 120; Hₚₐₜᵢₑₙₜ is a height of patient 706 from the floor; and H_{light} is the height of obstacle 710 from the floor. The above variables can be measured from any suitable reference frames, such as a common reference frame that is attached to a base of follower device 104. Zₛᵤₛ, Hₚₐₜᵢₑₙₜ, and H_{light} are absolute variables measured from the floor. Z₀, zᵢ, z_{fk_i}, and z_{spar_i} are relative variables measured from the top of set-up structure 204.

Constraints 1 and 2 are based on the ranges of motion of vertical shaft 714 and drop-link joints 802, respectively. Constraints 3-4 are used to avoid collisions with patient 706 and obstacle 710, respectively. Constraint 5 ensures that a longest instrument is removable from a cannula mount 702 of a manipulator arm 120.

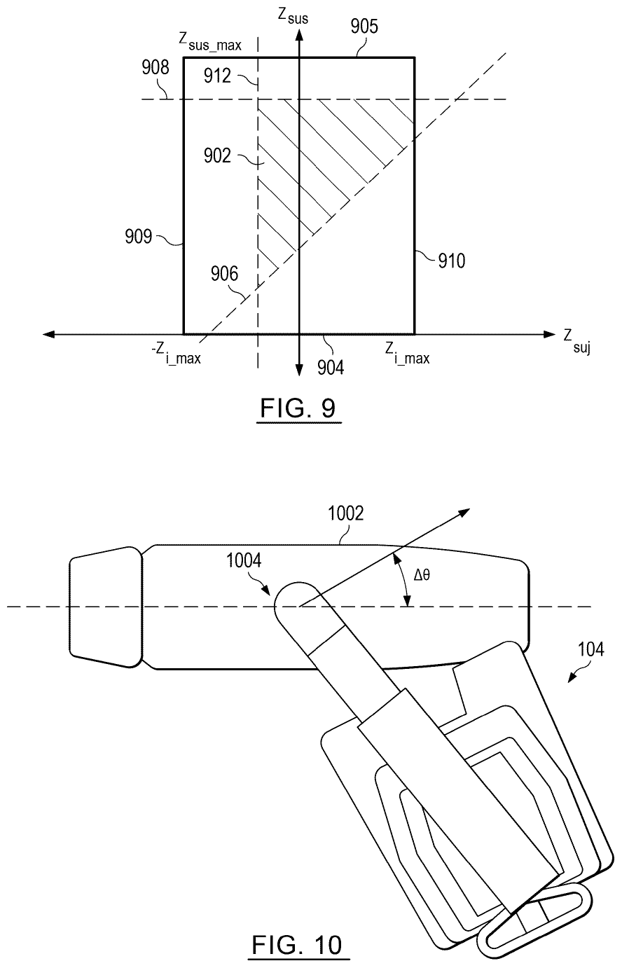

FIG. 9 illustrates an example feasible partitioning solution space for the example partitioning of FIG. 8, according to various embodiments. In some embodiments, a feasible solution space can be determined as the intersection of constraint surfaces for all DOFs/joints participating in a direction of interest. As shown, a feasible solution space 902 is the intersection of constraints surfaces 904-905 for a constraint associated with joint ROM limits that define the minimum and maximum heights of set-up structure 204 (constraint 1 in the above description of FIG. 8), which can vary between 0 and Z_{sus_max}: constraint surfaces 909-910 associated with joint ROM limits that define the minimum and maximum displacements of a drop-link joint 802 of the i^{th} manipulator arm 120 (constraint 2), which can vary between −z_{i_max} and z_{i_max}: a constraint surface 906 associated with a patient clearance constraint (constraint 3): a constraint surface 908 associated with a constraint for clearing obstacle 710 (constraint 4); and a constraint surface 912 associated with an instrument exchange constraint (constraint 5). In some embodiments, the constraints and associated constraint surfaces can be updated in real time.

In some embodiments, when the feasible solution space 902 includes multiple solutions, one solution can be selected using one or more cost functions. For example, the one or more cost functions can include cost functions based on displacements of joints of the repositionable structure to centers of ranges of motion of those joints, a measure of manipulability of links of the repositionable structure, a bandwidth of the DOFs, etc., or a combination thereof, as described above in conjunction with FIG. 6.

FIG. 10 illustrates an example of determining a motion of a repositionable structure system in an angular direction to approach an object, according to various embodiments. Similar to the discussion above in conjunction with FIG. 7, sensor data processing module 306 can use sensor data and a computer vision technique to determine the orientation of a table 1002. In addition, kinematics estimation module 308 can use kinematic data and kinematics to determine the orientation of a manipulator arm 120. Then, clearance estimation module 310 can determine an angular displacement between the orientation of table 1002 and the orientation of manipulator arm 120. Illustratively, the angular displacement can be the angle between a bearing angle of a midline of table 1002 and an angle of a cluster 1004 of manipulator arms 120 about an axis of the support structure 722, measured in a based frame of the follower device 104, shown as Δθ.

In addition, clearance estimation module 310 can determine angular recommended motion(s) of follower device 104 that increases (in repulsive cases) or decreases the angular displacement (in attractive cases) between the orientation of table 1002 and the orientation of the cluster 1004 of manipulator arms 120 based on a target angular displacement. As described, the target angular displacement can be a threshold angle plus a tolerance factor in some embodiments, and the target angular displacement can be different for different circumstances. In addition, the recommended angular motion(s) can be partitioned between multiple DOFs/joints that can move corresponding portion(s) of follower device (and/or other devices) in the angular direction of interest. For example, the recommended angular motion(s) could be partitioned between rotational joint 716 at the top of vertical shaft 714, rotational joint 720 coupling distal link 718 to support structure 722, and/or rotational joints 724 coupling manipulator arms 120 to support structure 722, described above in conjunction with FIG. 7.

Advantageously, techniques are disclosed that enable a computer-assisted system to be repositioned at a target position and/or orientation relative to a worksite while avoiding obstacles in the vicinity of the worksite. The disclosed techniques can decrease the likelihood that collisions with obstacles occur while also reducing the time needed to reposition the computer-assisted system at the target position and/or orientation. The disclosed techniques can also improve the range of motion of one or more working ends of the computer-assisted system at the target position and/or orientation, such as by retaining more ROM for joints used in a procedure performed at the target position and/or orientation in general, or in specific DOFs matched to the procedure.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted system comprising:
a repositionable structure system, the repositionable structure system comprising a plurality of links coupled by a plurality of joints; and
a control unit communicably coupled to the repositionable structure system,
wherein the control unit is configured to:
determine a target pose of a system portion of the computer-assisted system, the target pose of the system portion comprising at least one parameter selected from the group consisting of: a target position of the system portion and a target orientation of the system portion,
determine a current pose of the system portion, the current pose of the system portion comprising at least one parameter selected from the group consisting of: a current position of the system portion and a current orientation of the system portion,
determine a motion for the repositionable structure system based on a difference between the target pose and the current pose, the motion including a first component in a first direction,
determine a partitioning of the first component into a plurality of partitions, wherein a first partition of the plurality of partitions is associated with a first joint set of the plurality of joints, and a second partition of the plurality of partitions is associated with a second joint set of the plurality of joints, the first joint set differing from the second joint set, and
cause a first movement of the first joint set to achieve the first partition and a second movement of the second joint set to achieve the second partition.

2. The computer-assisted system of claim 1, wherein:
the computer-assisted system further comprises a sensor system configured to capture sensor data of an operating environment containing the repositionable structure system; and
the control unit is further configured to:
determine an object pose of an object portion of an object in the operating environment based on the sensor data, the object pose of the object portion comprising at least one parameter selected from the group consisting of: a position of the object portion and an orientation of the object portion; and
to determine the target pose, the control unit is configured to use the object pose.

3. (canceled)

4. The computer-assisted system of claim 1, wherein to cause the first movement and the second movement, the control unit is configured to perform at least one action selected from the group consisting of:
command an actuator system to move the first joint set and the second joint set;
output a first prompt perceivable by an operator of the computer-assisted system, the first prompt prompting the operator to move the first joint set to achieve the first partition; and
output a second prompt perceivable by the operator, the second prompt prompting the operator to move the second joint set to achieve the second partition.

5. The computer-assisted system of claim 1, wherein:
the first joint set consists of a single joint of the plurality of joints; or
the second joint set comprises multiple joints of the plurality of joints.

6. The computer-assisted system of claim 1, wherein to determine the partitioning of the first component into the plurality of partitions, the control unit is configured to:
determine a set of constraints associated with moving the system portion in the first direction; and
determine the partitioning based on the set of constraints.

7. The computer-assisted system of claim 6, wherein to determine the partitioning based on the set of constraints, the control unit is configured to:
compute one or more values of one or more cost functions associated with one or more partitioning candidates that satisfy the set of constraints; and
select the partitioning based on the one or more values;
wherein the one or more cost functions comprise at least one cost function selected from the group consisting of:
a cost function associated with a displacement of a joint of the first joint set or the second joint set to a center of a range of motion associated with the joint;
a cost function associated with a manipulability of a link of the plurality of links; and
a cost function associated with a bandwidth of a joint of the first joint set or the second joint set.

8. (canceled)

9. The computer-assisted system of claim 6, wherein to determine the partitioning based on the set of constraints, the control unit is configured to:
determine that no partitioning candidates satisfy the set of constraints; and
output a notification perceivable by an operator, the notification notifying the operator of an error or prompting the operator to change at least one constraint in the set of constraints.

10. (canceled)

11. The computer-assisted system of claim 6, wherein
the set of constraints comprises at least one constraint selected from the group consisting of: an average of a plurality of constraints, a least restrictive constraint of the plurality of constraints, and a most restrictive constraint of the plurality of constraints; and wherein
different constraints of the plurality of constraints are associated with different system portions of the computer-assisted system.

12. The computer-assisted system of claim 1, wherein the target pose of the system portion is a pose that separates an object pose of an object in an operating environment containing the repositionable structure system from the system portion by a target separation, the target separation comprising a target linear displacement or a target angular displacement.

13. The computer-assisted system of claim 12, wherein the control unit is further configured to determine or modify the target separation based on at least one parameter selected from the group consisting of:
an environmental feature of an operating environment containing the repositionable structure system;
an operating mode of the computer-assisted system;
an operating condition of the computer-assisted system;
a status of an operator of the computer-assisted system; and
an automatically determined operator preference.

14. (canceled)

15. The computer-assisted system of claim 1, wherein to cause the first movement of the first joint set to achieve the first partition, the control unit is configured to:
determine a speed based on at least parameter selected from the group consisting of: a displacement from the system portion to an object portion of an object in the operating environment, a speed at which the system portion is moving towards the object portion, a type of the object, and an automatically-determined operator preference; and
control the first joint set to move one or more links of the plurality of links based on the determined speed.

16-19. (canceled)

20. The computer-assisted system of claim 1, wherein to cause the first movement and the second movement, the control unit is configured to: simultaneously cause the first movement and the second movement.

21. (canceled)

22. A method for controlling a repositionable structure system comprising a plurality of links coupled by a plurality of joints, the method comprising:
determining a target pose of a system portion of a computer-assisted system, the target pose of the system portion comprising at least one parameter selected from the group consisting of: a target position of the system portion and a target orientation of the system portion;
determining a current pose of the system portion, the current pose of the system portion comprising at least one parameter selected from the group consisting of: a current position of the system portion and a current orientation of the system portion;
determining a motion for the repositionable structure system based on a difference between the target pose and the current pose, the motion including a first component in a first direction;
determining a partitioning of the first component into a plurality of partitions, wherein a first partition of the plurality of partitions is associated with a first joint set of the plurality of joints, and a second partition of the plurality of partitions is associated with a second joint set of the plurality of joints, the first joint set differing from the second joint set; and
causing a first movement of the first joint set to achieve the first partition and a second movement of the second joint set to achieve the second partition.

23-24. (canceled)

25. The method of claim 22, wherein causing the first movement and the second movement comprises performing at least one action selected from the group consisting of:
commanding an actuator system to move the first joint set and the second joint set;
outputting a first prompt perceivable by an operator of the computer-assisted system, the first prompt prompting the operator to move the first joint set to achieve the first partition; and
outputting a second prompt perceivable by the operator, the second prompt prompting the operator to move the second joint set to achieve the second partition.

26. The method of claim 22, wherein:
the first joint set consists of a single joint of the plurality of joints; or
the second joint set comprises multiple joints of the plurality of joints.

27. The method of claim 22, wherein determining the partitioning of the first component into the plurality of partitions comprises:
determining a set of constraints associated with moving the system portion in the first direction; and
determining the partitioning based on the set of constraints.

28. The method of claim 27, wherein determining the partitioning based on the set of constraints comprises:
computing one or more values of one or more cost functions associated with one or more partitioning candidates that satisfy the set of constraints; and
selecting the partitioning based on the one or more values;
wherein the one or more cost functions comprise at least one cost function selected from the group consisting of:
a cost function associated with a displacement of a joint of the first joint set or the second joint set to a center of a range of motion associated with the joint;
a cost function associated with a manipulability of a link of the plurality of links; and
a cost function associated with a bandwidth of a joint of the first joint set or the second joint set.

29-31. (canceled)

32. The method of claim 27, wherein
the set of constraints comprises at least one constraint selected from the group consisting of: an average of a plurality of constraints, a least restrictive constraint of the plurality of constraints, and a most restrictive constraint of the plurality of constraints; and
different constraints of the plurality of constraints are associated with different system portions of the computer-assisted system.

33. The method of claim 22, wherein;
the target pose of the system portion is a pose that separates an object portion of an object in an operating environment containing the repositionable structure system from the system portion by a target separation, the target separation comprising a target linear displacement or a target angular displacement; and
the method further comprises determining or modifying the target separation based on at least one parameter selected from the group consisting of:
an environmental feature of the operating environment;
an operating mode of the computer-assisted system;
an operating condition of the computer-assisted system;
a status of an operator of the computer-assisted system; and
an automatically determined operator preference.

34-42. (canceled)

43. One or more non-transitory machine-readable media comprising a plurality of machine-readable instructions which when executed by one or more processors are adapted to cause the one or more processors to perform a method for controlling a repositionable structure system comprising a plurality of links coupled by a plurality of joints, the method comprising:
determining a target pose of a system portion of a computer-assisted system, the target pose of the system portion comprising at least one parameter selected from the group consisting of: a target position of the system portion and a target orientation of the system portion;
determining a current pose of the system portion, the current pose of the system portion comprising at least one parameter selected from the group consisting of: a current position of the system portion and a current orientation of the system portion;
determining a motion for the repositionable structure system based on a difference between the target pose and the current pose, the motion including a first component in a first direction;
determining a partitioning of the first component into a plurality of partitions, wherein a first partition of the plurality of partitions is associated with a first joint set of the plurality of joints, and a second partition of the plurality of partitions is associated with a second joint set of the plurality of joints, the first joint set differing from the second joint set; and
causing a first movement of the first joint set to achieve the first partition and a second movement of the second joint set to achieve the second partition.
